(19) United States
(12) Patent Application Publication
(10) Pub. No.: US 2005/0288606 A1
(43) Pub. Date: Dec. 29, 2005

(54) Culture swab with protective cap and safety pin

(75) Inventor: Richard Raymond Alter, Delavan, WI (US)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(73) Assignee: Continental Plastic Corp

(21) Appl. No.: 10/877,255

(22) Filed: Jun. 25, 2004

(51) Int. Cl. A61B 10/00

(52) U.S. Cl. 600/572; 600/569

(57) ABSTRACT

A veterinary instrument for use in animal husbandry that includes an inner member with a swab or similar element attached at one end, located inside a second inner member meant to protect the innermost member and attached swab, and an outermost member that surrounds both inner members and has a closed mold manufactured protective cap on one end. The instrument has one or two safety pins which ensure that the instrument cannot be used until they are removed.

### BACKGROUND OF THE INVENTION

1. Field of the Invention

The present invention relates to a novel veterinary device or instrument useful in medication, antiseptic, culture collection, and artificial insemination processes utilized in animal husbandry. More particularly, the present invention relates to a veterinary instrument and sealed holder having improvements to the cap and the holding tube that make the device function more effectively and lower the cost and time of manufacturing.

2. Discussion of the Related Art

U.S. Pat. No. 4,586,604 to Alter and entitled “Culture Collection Instrument and Sealed Swab Holder Therefor” discloses one device in the prior art. U.S. Pat. No. 4,457,313 to Alter entitled “Shield Protector for Artificial Insemination and Culture Collection Instruments” discloses another. The disclosure of each of these patents in its entirety is hereby expressly incorporated by reference into the present application for the purposes of indicating the background of the present invention and illustrating the state of the art.

As is known to those skilled in the art, the collection of a sample from the cervix of animal requires that the culture collection device must first pass through the vulva and/or cervical canal of the animal. Of course, these are not sterile and generally contain bacteria or diseased germs, such as vaginal or uterine infections or microplasa. Thus, previously recognized problems have been that when the instrument is inserted into the animal the culture collection instrument can transport infectious contaminants from the vulva and/or cervical canal into the cervix of the animal, or the element that will ultimately collect the sample can be prematurely contaminated before reaching the area that is the actual subject of the culture collection. Needless to say, it is worthwhile to have a culture collection instrument that reduces the contamination of both the cervix of the animal and the culture collection element while the instrument is being inserted into the animal. The same can be said for artificial insemination instrumentation.

Despite past improvements in the art, prior designs did not completely eliminate the contamination problems associated with collecting a culture of insemination. While some devices did solve the problem of how to protect the element delegated to take the culture, they left room for material improvement in mitigating the threat of contamination of the cervix by the transportation of contaminants from outer parts of the animal's reproductive tract. Specifically in some prior art devices, the protective cap or tip on the end of the device was scored on the outside surface so as to allow the protected and sterile inner members to break through the protective cap once the instrument had reached the area where the sample was to be taken from. The scoring on the outside of the cap provided groves or channels that, in effect, acted as areas of entrapment for foreign material that could entrap material during both insertion and before the device was used. The scoring of the protective cap had been necessarily located on the outside of the cap because of the method of manufacture. The cap was produced by hand utilizing a repetitive dipping process and then later hand scoring the cap on the outside with a razor or similar instrument. The method of manufacturer of this cap resulted in a further disadvantage in that the hand made nature of the process was costly, time consuming, and unable to be automated, which all led to increased costs for the final culture collection device product.

These prior methods of production also led to a further undesired characteristic of the instruments in that the force required to push the inner members through the protective cap varied substantially among individual devices. This situation was a direct result of the fact that the method of manufacture of the hand produced caps led to variations in material thickness and depth of scoring among the caps. The inconsistent force required to employ the sample taking element frustrated ease of use because product purchasers could never become familiar with a consistent method of use.

What is needed, therefore, is a protective cap that does not have areas of entrapment of foreign material on the outside of cap and that has a consistent thickness and depth of scoring or perforation. Achieving these desired cap characteristics requires a new method of cap manufacturing that is consistent and that would ideally be cheaper, less time intensive, and compatible with automation.

An additional limitation of the prior art is that the devices could be prematurely deployed. For example, the inner members could break through the protective cap during transportation or handling prior to being inserted into an animal to collect a culture. When this occurs, the device is effectively useless because the sample collecting element is not sufficiently protected from contamination once the protected cap is breached.

Therefore, what is further needed is a method to prevent the inner elements from breaching the protective cap until that time as when the device user is ready to deploy the device to collect a sample.

### SUMMARY AND OBJECTS OF THE INVENTION

A primary object of the invention is to provide an improved culture collection device or insemination instrument that isolates and protects the inner portion of the device upon its insertion into or removal from the interior of the body or cervix of an animal and to sealingly retain this element for subsequent handling and transport to the analysis laboratory. Another objective of the invention is to incorporate a manufactured cap in the instrument that lessens the chance of contamination to the sample or tube and inner areas of the animal by eliminating areas of entrapment of foreign material on the device's protective cap and also makes collection of a sample easier by providing a protective cap medium that requires a consistent force to be breached when the swab is being applied to the sample area inside the animal. Another object of the invention is to make manufacture of such devices cheaper and more efficient by use of incorporating the manufactured cap into the device. A further object of the invention is to make transport, handling, and storage of the device safer by including at least one safety pin in the device, which prevents premature deployment of the sample taking members.

In one embodiment of the invention a first inner member, comprised of a swab at the first end attached to an elongated tube or rod, is inside a second inner member. The second inner member is further inside an outer member that is sealed at the first end with a protective cap perforated on the inside. The perforations are predetermined thin sections, such as in the shape of an “X”, incorporated into the piece with all slits or indentations located on the inside surface of the cap. Consequently, the outside of the cap is a smooth, continuous surface lacking any areas of entrapment that could possibly collect foreign matter before or while the instrument is inserted past the vulva lining and through the cervical canal and then transport it to the cervix or other sample area. To deploy the device and take the sample, the second inner member with the first inner member's swab end inside, is pushed forward so that the first end of the second inner member breaches the cap of the outer member. The first inner member is then pushed forward through the open first end of the second inner member so that the swab extends past the first end of the second inner member, thereby being introduced to the environment to be sampled.

Once the sample is taken, the swab is retracted back into the second inner member and the second inner member, with the first inner member inside, is retracted back to its original position inside the outer member. After the swab and second inner member are again isolated inside the outer member, the entire device is removed from the animal. The swab, now containing the sample, is then preserved for testing by removing the second inner member and the enclosed first inner member from the outer tube and then capping the open first end of the second inner member, which is no longer enclosed by the outer member. The second inner member, which protects the swab of the first inner member, has a scored section, located bellow the point where the swab inside is positioned, that completely circumvents the second inner member and allows for the second inner member to be easily and cleanly broken at the scored section. The enclosed elongated tube or rod of the first inner member also has a scored section that is aligned with the scored section of the second inner member while the device is in its retracted position, and this scored section allows the first inner member to break contemporaneously with, and at the same point, as the second inner member when second inner member is broken. The broken end of the second inner member is then capped on the broken end so that it is now capped on both ends and contains the broken off end of the first inner member and its swab element inside. The user now has a sealed and sanitary holder for the swab element that prevents contamination of the sample and facilitates transportation and storage for later testing.

Additionally, a preferred embodiment also can have one or more safety pins that prevent the first inner member, second inner member, and outer member from moving longitudinally in relation to each other before the safety pins are removed. These pins are meant to protect from the inner members shifting in a manner that would permit the second inner member, and enclosed first inner member, to breach the outer member's protective cap before the user is ready to deploy the device to collect a sample. The use of these safety pins makes shipping and handling of the items safer and ensures the device cannot be employed until the pins are removed. This feature is important as the device is only good for one use, and the device must be discarded if the protective cap is breached while the instrument is outside of the animal.

In another embodiment of the invention, the device may be used as an artificial insemination instrument. In this embodiment the swab element of the first inner member is not present and the first inner member is simply a tube or solid rod, which is substantially the same size as the inner diameter of the second inner member. The semen sample is placed inside the second inner member in the cavity which exists between the end of the first inner member and the end of the second inner member. Once the device is fully inserted into the animal and the second inner member breaks through the protective cap, the first inner member is pushed forward to at least the end of the second inner member, thereby discharging the semen sample into the cervix of the animal. The device is then withdrawn from the animal and discarded.

In another embodiment of the invention, the cap on the end of the outer member is a molded plastic cap manufactured in a mold process, such as an injection molding process, with predetermined perforations, such as in the shape of an “X”, incorporated into the cap. The mold is designed so that any slits or indentations of the perforations are molded onto the inside surface of the cap, thereby leaving the outer surface of the cap a smooth, continuous surface. The cap can be manufactured in a reproducible process that assures consistent thickness of the normal and the perforated sections of the cap. The precise manufacturing method of the protective cap, and the uniform perforations that result, ensure that the force required to push the second inner member through the cap is consistent between different devices.

In another embodiment of the invention, the one or two safety pins consist of a loop sufficient in size to accommodate a human finger and a pin like projection extending from that ring. Preferably, the first inner member, second inner member, and outer member all have a pair of identical holes that are oriented perpendicular to the axis of the members and are located in such a manner that on each member each hole directly opposes its paired hole. When the instrument is in its predeployed state all three pairs of the holes align collinearly so that the pin's projection, which is longer than the diameter of the outer member, can pass through all three pairs of holes, thereby physically preventing the members from shifting longitudinally in relation to each other. To enable the instrument for use the user must remove the pin or pins by first removing the pin tab, located near the end of pin projection, that prevents the pin from being removed and then grab hold of or slip his finger into the pin ring and pull the safety pin away from the instrument until it is removed.

In another embodiment of the invention, the ring of the first safety pin is connected to the projection of the safety pin so that the plane of the ring is at about a forty-five degree angle in relation to the projection. This configuration allows the operator of the device to grip and use the device like a syringe when pushing the second inner member through the protective cap. The user puts his thumb through the pin ring and then puts his index and middle fingers on the first flange so that the device is between the two fingers. By bringing his thumb and index and middle fingers together as if using a syringe, the operator can more easily force the second inner member to break through the protective end cap while deploying the device. This embodiment is especially useful when the device is for artificial insemination.

In one method of using the device, the device must be removed from a gas sterilized package before it is employed.

In another method of using the device, the device is first unwrapped from a gas sterilized package and then the safety pin or pins are removed. Subsequently the instrument is inserted into the cervical canal of a mare and when the device is in the desired spot the second inner member is pushed through the cap perforated with an “X”. The swab element is then pushed about two inches beyond the first end of the second inner member to collect the sample.

### BRIEF DESCRIPTION OF THE DRAWINGS

FIG. 1 is a perspective view of a culture collection instrument in accordance with the present invention;

FIG. 2 is a longitudinal cross-sectional view of the culture collection device of FIG. 1 in accordance with the present invention during insertion of the device into the cervical canal of an animal and before inner members have breached the protective cap;

FIG. 3 is a fractional longitudinal view of the culture collection device in accordance with the present invention showing the device fully deployed to take a sample while in the cervix of the animal with a protective cap breached by the first and second inner members and with a swab element extended beyond a first end of a second inner member so as to contact the sample target;

FIG. 4 is a fractional longitudinal view of the second inner member, after extraction from the animal and removal of an outer member, with the swab element retracted back inside the second inner member having successfully collected a sample and showing the subsequent breaking off of the first and second inner members along the aligned scored sections of the first and second inner members;

FIG. 5 is a longitudinal view of the broken off end of the second inner member capped on both ends so as to create a protective sealed environment for the broken off end of the first inner member, with the sample contained on the swab end of the first inner member;

FIG. 6 is a longitudinal cross-section view of a protective cap showing the smooth outer surface and perforated inner surface;

FIG. 7 is an axial rear view of the protective cap showing molded “X” perforations on an inside surface of the cap;

FIG. 8 is an axial front view of the protective cap showing the smooth outer surface and molded “X” perforations on the inside surface;

FIG. 9 is a side view of a safety pin for the culture collection device showing the pin like projection with associated tab and a ring shaped finger hold;

FIG. 10 is a longitudinal side view showing two safety pins inserted into the culture collection device.

FIG. 11 is a longitudinal cross sectional view showing mold pieces of an injection molding process with an inner mold piece having an “X” shaped protrusion that will create the “X” shaped perforations on the resulting molded end cap.

FIG. 12 is a fractional cross-sectional side view of a culture collection device with an alternative first safety pin where the pin ring is at about a 45 degree angle in relation to the pin projection.

FIG. 13 is a fractional cross-sectional bottom view of the culture collection device with an alternative first safety pin where the pin ring is at a 45 degree angle in relation to the pin projection.

FIG. 14 is a fractional cross-sectional side view of the veterinary device for use as an artificial insemination instrument wherein the swab element is not present and there is a semen sample inside the second inner member.

In describing the preferred embodiment of the invention which is illustrated in the drawings, specific terminology will be resorted to for the sake of clarity. However, it is not intended that the invention be limited to the specific terms so selected and it is to be understood that each specific term includes all technical equivalents which operate in a similar manner to accomplish a similar purpose. For example, the word attached or terms similar thereto are often used. They are not limited to direct connection but include connection through other elements where such connection is recognized as being equivalent by those skilled in the art.

### DESCRIPTION OF PREFERRED EMBODIMENTS

The present invention and the various features and advantageous details thereof are explained more fully with reference to the non-limiting embodiments described in detail in the following description.

The construction of a culture collection device, such as disclosed in the present inventor's prior U.S. Pat. No. 4,586,604 and U.S. Pat. No. 4,457,313 is well-known to those skilled in the art and therefore a detailed description thereof is not necessary to fully understand the present invention, which is directed to novel improvements in the construction of the protective cap 16 and the addition of one or more elements, specifically on or more safety pins 30 and 31.

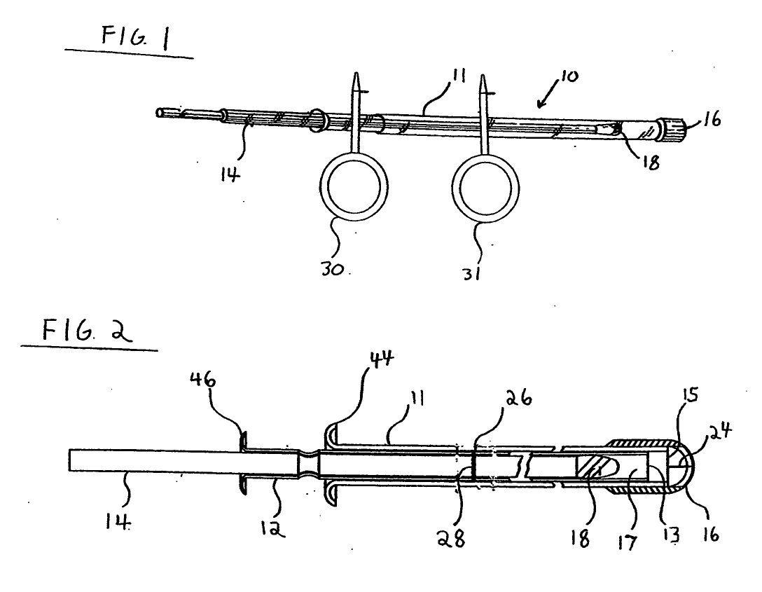

The invention is shown in FIGS. 1 and 2 which depict a veterinary device or instrument 10 that is comprised of an outer member 11, preferably a plastic tube, which has a protective cap or tip 16, preferably a injection molded plastic cap, on its first end. When used as a collection device 100, the device is further comprised of a first inner member 14 made up of a swab element 18 on the first end attached to an elongated rod or tube 19, preferably made of plastic. The culture collection device 100 is further comprised of a second inner member 12, preferably a plastic tube, which protects the first inner member 14. The culture collection device 100 also contains one or more safety pins 30 and 31 running through the second inner member 12, the first inner 14, and possibly the outer member 11.

Preferably, the culture collection device 100 is approximately 30 inches long and with a diameter of 1 inch. In the preferred embodiment, the outer member 11 is approximately 22 inches in length, the second inner member 12 is approximately 25 inches in length, and the first inner member 14 is approximately 30 inches in length. Preferably, the plastic used for the first inner member 14, second inner member 12, and outer member 11 is a softer PVC plastic and the swab element 18 is a polystyrene cotton swab.

As shown in FIGS. 1, 2 and 3, the cultural collection device 100 includes a second inner member 12 having a flange 46 at one end thereof, with the second inner member 12 adapted for movement relative to the outer member 11. The first inner member 14 is slidably positioned within the second inner member 12 and adapted for relative movement with respect to the second inner member 12. The first inner member 14 is constituted by a swab element 18, which may or may not be contained in a medium 17, located on the first end thereof and attached to an elongated rod or tube 19. Importantly, as best shown in FIGS. 2 and 4, the second inner member 12 includes a scored section 26 which, when the device 100 is in its predeployed state, is located forward the flange 44 of the outer member 11 and before the swab element 18, and further lines up with a scored section 28 on the elongated rod element 19. Upon the flexing of the second inner member 12 and the first inner member 14, the two scored sections 26 and 28 create a predetermined breaking point of the second inner member 12 that coincides with a predetermined breaking point of the first inner member 14, respectively.

To prepare for use of the device, the culture collection device 100 must first be removed from gas sterilized packaging (not shown). Next, the one or two safety pins 30 and 31 must be removed so that the first inner member 14, the second inner member 12, and the outer member 11 can all move longitudinally in relation to each other, as is required for the device to be deployed. The first pin 30 passes through the first inner member 14 and the second inner member 12 and is located forward of the flange 46 at the second end of the second inner member 12 and behind the flange 44 of the outer member 11, as shown in FIG. 10. Before it is removed the first pin 30 passes through the two opposing holes 52 and 53 in first inner member 14 and the two opposing holes 54 and 55 in the second inner member 12, with all four holes 52, 53, 54 and 55 being collinearly aligned. The second pin 31 passes through the first inner member 14, second inner member 12, and the outer member 11 and is located forward of the flange 44 at the second end of outer member 11 and behind the scored portion 26 of the second inner member 12, as shown in FIG. 10. While inserted the second pin 31 passes through the two opposing holes 32 and 33 in the first inner member 14, the two opposing holes 34 and 35 in the second inner member 12, and the two opposing holes 36 and 37 in the outer member 11, with all six holes 32, 33, 34, 35, 36 and 37 being collinearly aligned.

The process to remove a pin is the same for the first pin 30 and the second pin 31. To remove the first safety pin 30, the pin tab 42, located near the end of the pin projection 43 of the safety pin 30, must first be broken off or otherwise removed. With the pin tab 42 removed, the user then grabs hold of the pin ring 38 and pulls the pin ring 38 away from the culture collection device 10 so that the pin projection 40 travels through the collinearly aligned holes 52, 53, 54, and 55. The first safety pin 30 is fully removed from the culture collection device 100 when the end of the pin projection 43 passes through hole 55. Once pins 30 and 31 are removed, the culture collection device 11 is ready to be inserted into the animal in order to take a sample.

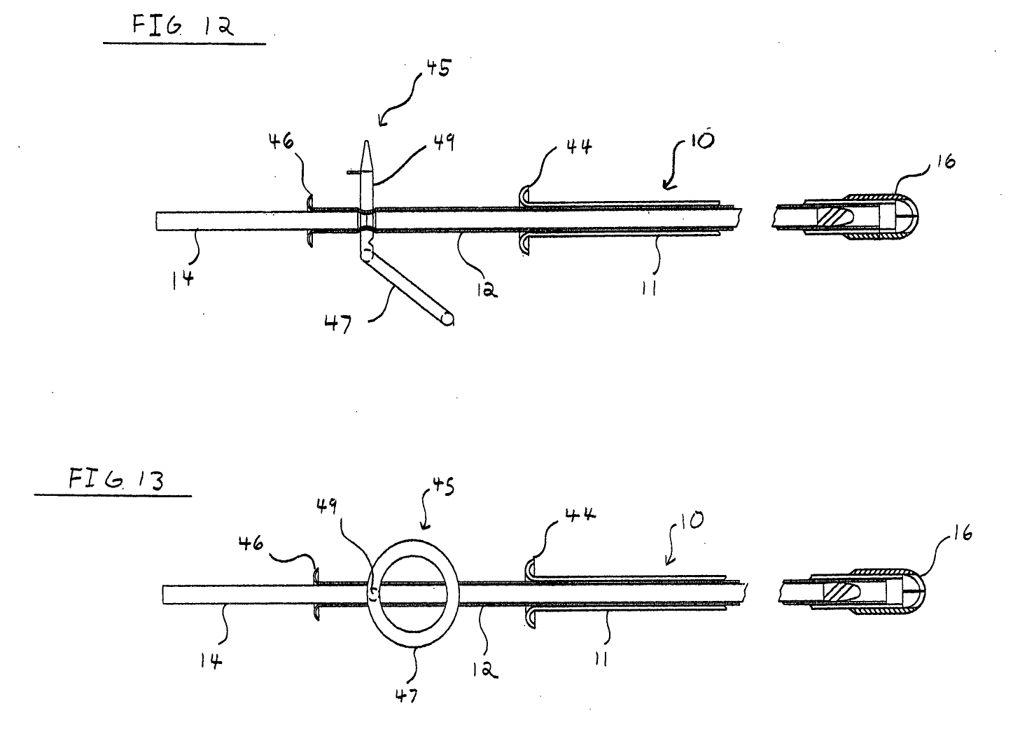

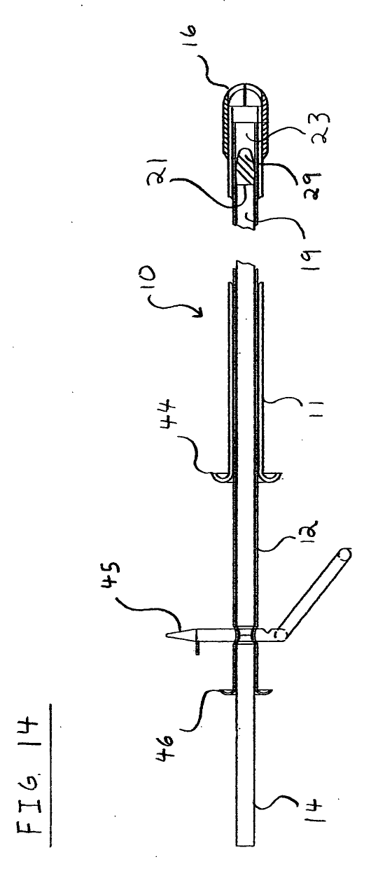

In one embodiment of the invention, the ring 47 of an alternate version of the first safety pin 45 is connected to the projection 49 of the safety pin 45 so that the plane of the ring 47 is at a forty-five degree angle in relation to the projection 49, as shown in FIGS. 12 and 13. This configuration allows the operator of the device to grip and use the device like a syringe when pushing the second inner member 12 through the protective cap 16. The user puts his thumb through the pin ring 47 and then puts his index and middle fingers on the first flange 44 so that the outer member 11 is between the two fingers. By bringing his thumb and index and middle fingers together as if using a syringe, the operator can more easily force the second inner member 12 to break through the protective cap 16 in order to deploy the veterinary device 10. This embodiment is especially useful when the device 10 is an artificial insemination instrument 110, as shown in FIG. 14.

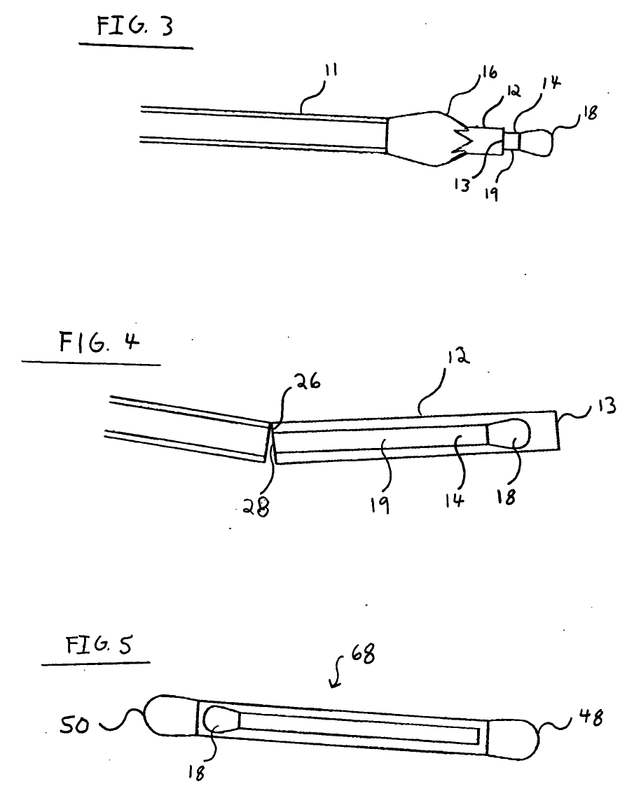

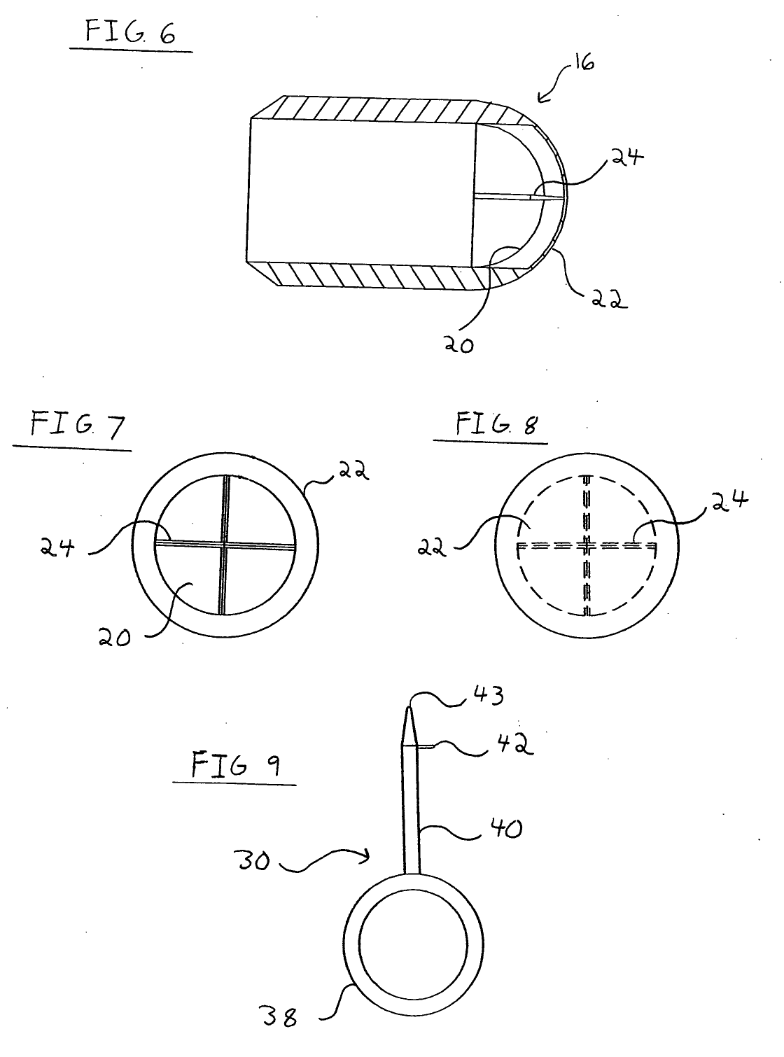

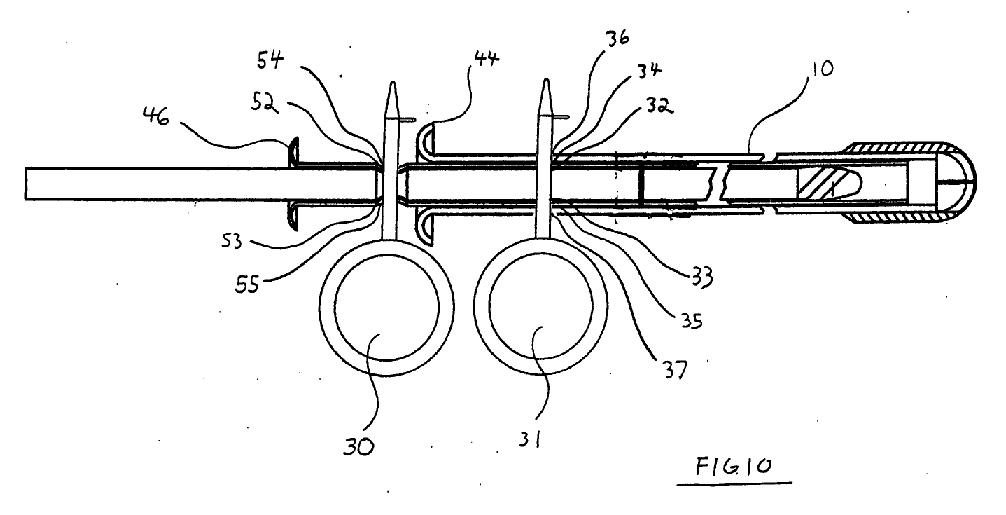

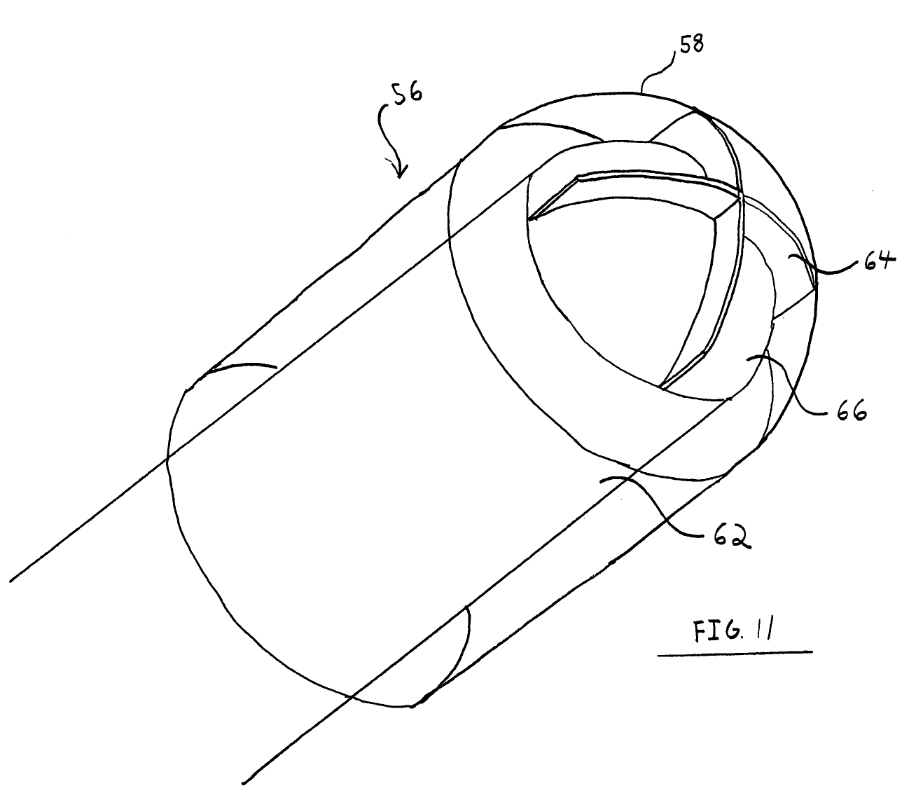

When the cultural collection device 100 is fully inserted into the cervix of an animal (not shown), the first inner member 14 is moved forward to engage the protective cap 16, which has an “X” shaped perforation 24 on the inner surface 20 of the cap 16, that breaks open upon engagement by the first end 13 of the second inner member 12, as shown in FIG. 3. The first inner member 14 is then pushed forward into the cervix of the animal, past the first end 13 of the second inner member 12 (or about 2 inches), so that the swab element 18 may either culture or apply medication to the animal, as desired, the position of final deployment as shown in FIG. 3.

Referring to FIGS. 3-5, upon completion of the culturing or medication processes, the first inner member 14 and the attached swab element 18 are retracted back inside the first end 13 of the second inner member 12. The first inner member 14 and the second inner member 12 are subsequently retracted together back inside the first end 15 of the outer member 11 so that the relative position of the three members 11, 12, and 14 is the same as it was before deployment, with the significant difference being the protective cap 16 has been irreversibly breached. Now that the culture collection device 100 is in its fully retracted position, the entire culture collection device 100 is removed from the animal.

The second inner member 12, containing the first inner member 14, is now withdrawn from the outer member 11, the position as partially shown in FIG. 4. A first end cap 48 is inserted upon the first end 13 of the second inner member 12 to seal one end of the second inner member 12.

Upon the manual flexing of the second inner member 12, the second inner member 12 readily and predeterminately breaks and separates at its scored section 26. As the flexing force translates through to the first inner member 14, the elongated rod element 19 of the first inner member 14 also readily and predeterminately breaks at its scored section 28, as shown in FIG. 4. By scoring, it is meant that the second inner member 12 and the first inner member 14 may have a notch or indentation either partially or completely therearound, which will permit a complete and clean breaking and division of the second inner member 12 and first inner member 14 upon manual flexing of the two. Upon the predetermined breaking of the second inner member 12 and the first inner member 14, a second end cap 50 is inserted upon the end of the swab protector element 12 opposite the first end 13 to provide a tubular sealed enclosure 68, which provides a sealed and sanitary holder for the cultured swab element 18. This tubular sealed enclosure 68 permits safe handling, storage, and transportation of the cultured swab element 18 prior to medical testing of the sample, as shown in FIG. 5.

In the embodiment of the invention for use as an artificial insemination instrument, the swab element 18 of the first inner member 14 is not present and the first inner member 14 is simply the elongated rod 19, which is substantially the same size as the inner diameter of the second inner member 12, as shown in FIG. 14. The semen sample 29 is placed inside the second inner member 12 in the cavity 23 that exists between the end of the elongated rod 19 and the first end 13 of the second inner member 12. Once the device 10 is fully inserted into the animal and the second inner member 12 breaks through the protective cap 16, the first inner member 12 is pushed forward to at least the first end 13 of the second inner member 12, thereby discharging the semen sample 29 into the cervix of the animal. The device is then withdrawn from the animal and discarded.

A substantial, novel improvement of this invention over the prior art results from the superior method of manufacturing the protective cap 16. Instead of being produced by hand in an open mold, as in the prior art, the culture collection device 10 of the present invention uses a manufactured protective cap 16 that is created in a closed molding process, such as an injection molding process. In the injection molding process, the mold 56 consists of a mold, made an outer shell 58 that defines the structure of the outer surface 22 of the cap 16 and a solid inner mold piece 62 that defines the structure of the inner surface 20 of the cap 16, as best seen in FIG. 11. The outer shell 58 of the mold 56 is preferably made of two mirrored halves that come together during the molding process to form the outer shell 58. The inner mold piece 62 has a “X” shaped protrusion 64 on the top end 66 of the inner mold piece 62 that results in the creation of the “X” shaped perforation 24 on the inner surface 20 of the protective cap 16 when the mold 56 is filled and the protective cap 16 is made. Preferably, the “X” shaped protrusion 64 on the inner mold piece 62 is situated such that the seams of the perforation 24 in the molded cap 16 will be offset from any seams created in the molded cap 16 by the transition between the two halves of the outer shell 58 of the mold 56.

The incorporation of the manufactured protective cap 16 results in superior performance of the culture collection device 100 for two reasons as discussed in the summary. First, the resulting smooth outer surface 22 of the cap 16 does not contain any slits or channels that can entrap foreign matter and then transport it into the cervix of the animal. Second, because of the precise nature of the molding process, the thickness of the cap 16 and the depth on the perforation 24 in the inner surface 20 of the cap 16 are uniform between devices, so that the user is required to apply the same force in order to breach the protective cap 16 with each use of a device, which promotes efficiency and ease of use. Further, the molding process for manufacturing the protective cap 16 is advantageous because it is cheaper than producing the protective cap in an open mold process and the closed mold process can be automated and easily reproduced, which further reduces the costs and time of manufacture and increases product performance

The above-illustrated specific embodiments of the present invention are capable of variation and modification, and, therefore, the examples are intended merely to facilitate an understanding of ways in which the present invention may be practiced and to further enable those of skill in the art to practice the present invention. Accordingly, the examples should not be construed as limiting the scope of the present invention.

1. A veterinarian device comprising:
a) an inner tube;
b) a second inner tube in communication with the first inner tube;
c) an outer tube in communication with a first and second inner tube; and
d) a cap attached to a first end of the outer tube having an inner perforated section.

2. The device of claim 1, further comprising a safety pin having first end with a loop and a second end with a projection perpendicular to the axis of the first and second tubes.

3. The device of claim 1, further comprising a second safety pin closer to a second end of the first inner tube.

4. The device of claim 1, wherein the cap has a perforation in the shape of an X molded into the inside surface of first end.

5. The device of claim 1, further comprising a medium in the second inner tube.

6. The device of claim 1, wherein the cap is manufactured from plastic and from a mold containing a projection to the shape of an X.

7. The device of claim 1, wherein the first inner tube includes a first end having a swab and a second end having a flanged portion.

8. The device of claim 1, wherein the second inner tube includes a second end having a flanged portion.

9. The device of claim 1, wherein the outer tube includes a second end having a flanged portion; and wherein the first and second tubes are scored to be easily reduced in length after the culture is collected.

10. The device of claim 1, further comprising a first and second molded end cap attachable to the second inner tube.

11. A veterinarian device comprising:
a) a first inner member for collecting a culture;
b) a second inner member for receiving the first inner member;
c) an outer member for receiving the first and second inner member; and
d) a molded plastic cap for sealing a first end of the outer member until it can be broken through to release the first and second inner members.

12. The device of claim 11, further comprising a first safety pin to lock the first and second member together and prevent them from penetrating the cap during shipment.

13. The device of claim 11, further comprising a second safety pin for locking the first and second inner members initially together so that when pushed toward they travel together.

14. The device of claim 11, further comprising a perforation in the shape of an “+” molded into cap.

15. The device of claim 11 further comprising a medium for preserving the culture collected on the inner member until the culture can be tested.

16. The device of claim 11, wherein the cap is made in a mold containing a projection in the shape of an “+”.

17. The device of claim 11, wherein the first inner member includes a first end for collecting a culture and an enlarged second end for better leverage when the member is pushed.

18. The device of claim 11, wherein the second inner member includes an enlarged end for better leverage and stability when the member is pushed.

19. The device of claim 11, wherein the outer member includes a flanged portion for better grip when the inner members are pushed.

20. The device of claim 11, wherein the first and second members are easily broken and then sealed with a molded plastic first cap and second cap.

21. An instrument used for the collection of samples from an animal comprising:
a) an inner member having an absorbent material at a first end section, a scored middle section, and an first alignment hole at a third end section and opposite the first end section, wherein the hole is perpendicular to a longitudinal axis of the member;
b) a second tubular member having a radial aligned with scored section of the inner member, and a second alignment hole perpendicular to the longitudinal axis of the second member and alignable with the first hole in the first member;
c) third tubular member for encapsulating the first and second members and wherein the third member has a hole alignable with the first and second holes;
d) a pin for inserting into the holes in the first, second, and third members, the pin having a loop of sufficient size to accept a human finger and a projection radiating therefrom and having a length greater than the diameter of the holes, the projection including a tab; and
e) a cap of sufficient diameter to fit over the third member allowing the first and second member to be contained within the third member, the cap having internal relief segments capable of being released with longitudinal pressure from the first and second members.

22. A method of using a veterinary instrument for collecting a culture from an animal comprising the steps of:
A. unwrapping a gas sterilized packaging;
B. removing a retaining pin;
C. pushing two inner members against a cap of a third member thereby breaking relief segments in the cap;
D. allowing the segments to protrude from the capped end of the third member;
E. moving the first member further longitudinally as to project from the second inner member and thereby to come into contact with the sample area to be taken;
F. moving the first inner member in the reverse direction until it is once again enclosed in the second member;
G. removing the instrument from the animal;
H. removing the first and second member from the third member;
I. breaking the first and second members at a radial score section of the second member; and
J. capping on both ends of the now shortened second member.

23. A method for collecting a culture from a brood mare comprising the steps of:
A. providing a culture collection unit comprising of an outer sheath, inner sheath and swab;
B. inserting the unit into a cervical canal of the mare,
C. pushing the inner sheath through an “x” shaped perforation molded into a enclosing tip of the outer sheath;
D. sliding the swab through the inner sheath;
E. obtaining the culture;
F. retracting the swab back into the inner sheath;
G. removing the inner sheath and swab from the outer sheath;
H. discarding the outer sheath;
I. capping a first exposed end of the inner sheath containing the culture;
J. snapping the sheath and swab at pre-scored lines;
K. capping the second end of the sheath; and
L. forwarding the culture for analysis.

24. A method for producing a protective cap for a veterinary device using injection molding comprising;
A. providing an outer mold shell made up of two mirrored halves;
B. providing a inner mold piece made up of a solid piece with an “X” shaped protrusion on the top that is aligned so as to ensure the resulting “X” shaped perforations in the mold will not be aligned with the flanges created at the meeting of the two halves of the outer mold shell;
C. closing the mold to create a hollow shape inside the closed mold that represents the precise dimensions of the desired protective cap; and
D. using the mold to create the designed protective cap using standard injection molding techniques and technologies.
